(19) United States
(12) Patent Application Publication
(10) Pub. No.: US 2013/0123364 A1
(43) Pub. Date: May 16, 2013

(54) N6-(1-IMINOETHYL)-L-LYSINE FOR REGENERATION OF ALVEOLI IN LUNGS

(71) Applicant: Norbert Weissmann, Pohlheim (DE)

(72) Inventor: Norbert Weissmann, Pohlheim (DE)

(21) Appl. No.: 13/737,774

(22) Filed: Jan. 9, 2013

Related U.S. Application Data
(63) Continuation-in-part of application No. PCT/EP2011/061474, filed on Jul. 7, 2011.

(51) Int. Cl. A61K 31/198

(52) U.S. Cl. CPC A61K 31/198
USPC 514/564; 562/560

(57) ABSTRACT

The present invention describes the use of N⁶-(1-iminoethyl)-L-lysine for the manufacture of a medicament for the treatment of pulmonary alveolar damage or destruction in mammals, including in humans.

### CROSS-REFERENCES TO RELATED APPLICATIONS

This Application is a continuation-in-part of PCT Application No. PCT/EP2011/061474, filed Jul. 7, 2011, which is incorporated herein by reference in its entirety as if fully set forth herein.

### FIELD OF THE INVENTION

The present invention concerns the use of N⁶-(1-iminoethyl)-L-lysine for the manufacture of a medicament for the regeneration of alveoli in the lung.

### BACKGROUND OF THE INVENTION

The main function of the lung is to supply the blood with oxygen and to simultaneously dispose carbon dioxide. This happens in alveoli, an anatomical structure in the lung parenchyma, being the terminal end of the respiratory tree. Alveoli are particular to mammalian lungs. The alveolar membrane is the gas-exchange surface. Carbon dioxide rich blood is pumped from the rest of the body into the alveolar blood vessels where it through diffusion releases its carbon dioxide and absorbs oxygen.

Intoxication, shear stress, air pollution, or infectious agents can damage and destroy alveoli, thereby inducing life-threatening condition for the host, as the lungs cannot spontaneously regenerate alveolar structure. Consequently, the patients may require lung transplantation. Mortality rates associated with lung transplantations are high. Therefore, a high medical need exists for a medicament suitable to regenerate the damaged or destroyed alveoli. To date, no such medication is available.

N⁶-(1-iminoethyl)-L-lysine (L-NIL) is a water-soluble compound (CAS 159190-45-1 or CAS 150403-89-7) having the following chemical formula:

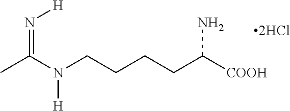

### SUMMARY OF THE INVENTION

An aim of the present invention is to overcome the disadvantages of the state of the art and to provide an agent which is suitable for manufacture of a medicament for the regeneration of destroyed alveoli. The problem is solved according to the present invention by utilizing L-NIL for the manufacture of a medicament for the regeneration of alveoli according to the claims.

### BRIEF DESCRIPTION OF DRAWINGS

FIG. 1A and FIG. 1B illustrates the curative regeneration of alveolar destruction and vessel remodeling in lung after toxic tobacco smoke exposure by L-NIL treatment in mice;

FIG. 2 gives the total number of alveoli assessed by quantitative stereology;

FIGS. 3A to 3D show representative slides of elastin-stained lungs;

FIG. 4 depicts lung compliance after 8 months of toxic smoke exposure in mice; and

FIGS. 5A to 5D shows the induction of proliferation and the regeneration of alveoli by treatment with L-NIL for 3 month after previous lung exposure to toxic smoke for 8 months.

### DETAILED DESCRIPTION OF EXEMPLARY EMBODIMENTS

Surprisingly it was found that in mouse lungs, which were damaged by intoxication with tobacco smoke, or by other structural alterations, a regeneration of the lung alveoli is achieved. In addition, this structural restoration led to a restoration of the function of the lung alveoli.

Therefore, L-NIL is a suitable agent for the manufacture of a pharmaceutical agent for the regeneration of the damaged or destroyed lung alveolar structure and function.

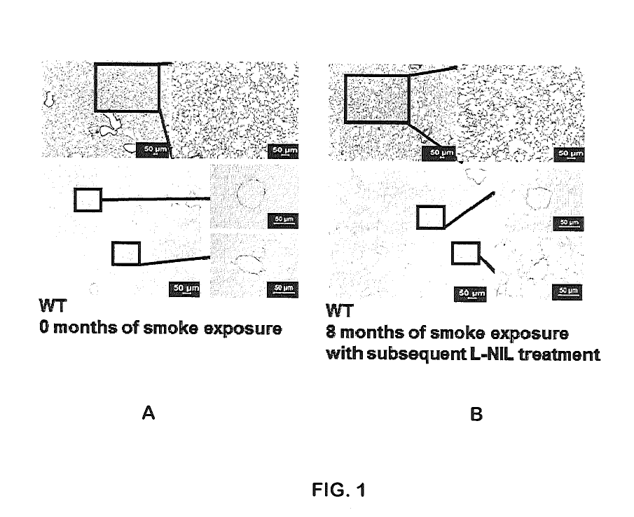

After oral administration of L-NIL in the drinking water in a concentration of 600 μg/ml, mice show after 3 month a regeneration of the damaged or destroyed lung alveoli. FIG. 1A and FIG. 1B illustrates the curative regeneration of alveolar destruction and vessel remodeling in lung after toxic tobacco smoke exposure by L-NIL treatment in mice.

These results can be extrapolated to other mammals, including humans, as all mammalian lungs have very similar alveolar structures.

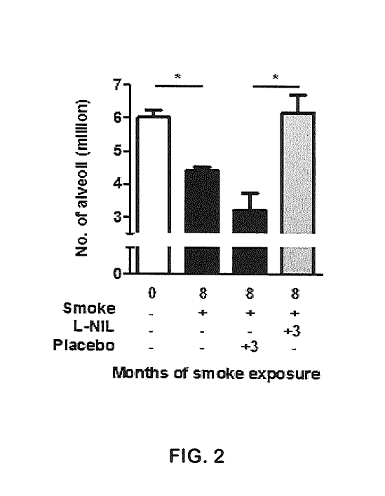

FIG. 2 gives the total number of alveoli assessed by quantitative stereology. The figure illustrates regeneration of alveoli after toxic tobacco smoke exposure by L-NIL treatment in mice.

L-NIL is therefore used according to this invention as agent for the manufacture of a medicament for the treatment of damaged or destroyed pulmonary alveoli in mammals, including human patients.

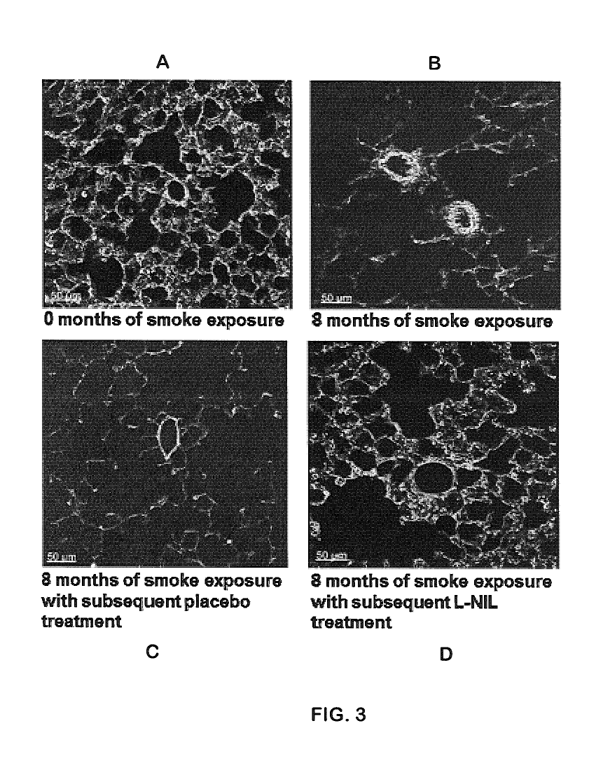

FIGS. 3A to 3D show representative slides of elastin-stained lungs. The figure demonstrates the regeneration of the elastic fibers network by L-NIL treatment in mice after destruction by toxic tobacco smoke exposure in mice.

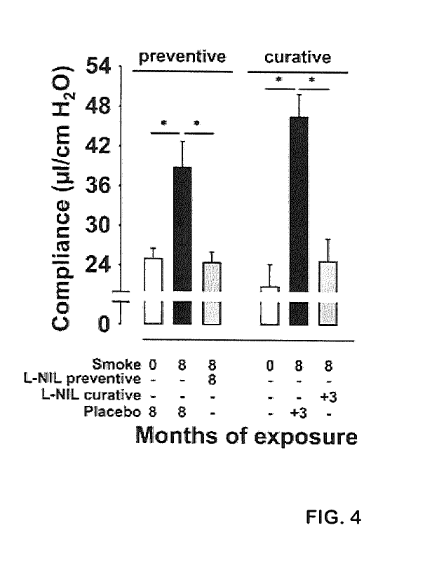

FIG. 4 depicts lung compliance after 8 months of toxic smoke exposure in mice. The lung function is measured in isolated perfused and ventilated lungs after explantation (negative pressure ventilation). The treatment with L-NIL is either carried out in parallel to the smoke exposure over 8 months (preventive, left), or after termination of smoke exposure (8 months of smoke exposure), followed by L-NIL or placebo treatment for additional 3 months (curative, right). For comparison, non-smoke-exposed animals (0 months) are shown. Statistically significant differences (P<0.05) as compared with placebo treatment are indicated by asterisks.

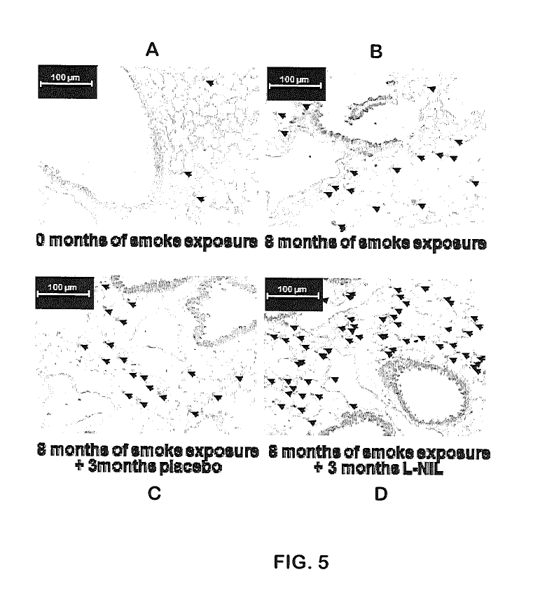

FIGS. 5A to 5D shows the induction of proliferation and the regeneration of alveoli by treatment with L-NIL for 3 month after previous lung exposure to toxic smoke for 8 months. Arrows indicate cells, which are positive for the proliferation marker PCNA. Depicted are lung sections of non-smoking animals (0 months) and lungs of animals, which were, after 8 months of toxic smoke exposure, treated either with L-NIL or with placebo for 3 months.

The term patient thereby equally refers to humans and other mammals. The agent can thus be applied in human and in veterinary medicine. The L-NIL-containing medicament of this invention is administered to the patient as part of a pharmaceutically acceptable composition either orally, by inhalation, or by injection. The physician must determine the therapeutic dosage for alveolar regeneration.

The agent of this invention is preferably administered as a medicament orally as aqueous solution or in tablet form. Particularly preferred is the administration of the agent of this invention as a medicament using a standard aerosol inhalation procedure by nebulizer or inhaler. As aqueous solution, the agent of this invention is preferably nebulized with suitable commercially available piezoelectric, jet-, ultrasonic aerosol generators or soft-mist inhalers and nebulizers. Examples for commercially available nebulizers are: jet nebulizers such as Bennett-Raindrop., Pan LC., Pan LL., Ventstream., ultrasonic nebulizers such as Multisonic pro., Pulmosonic5., Systam LS, or metered dose inhalers.

The deposition of aerosols in the respiratory tract depends on the particle size distribution of the aerosol. The agent of this invention is preferably used in the form of particles with a mass median aerodynamic diameter of less than 6 micrometers, to reach the damaged alveoli.

Pharmaceutically acceptable compositions may include modifications e.g. as salts, esters, or amides.

### WORKING EXAMPLES

In vivo Studies with L-NIL for the Treatment of Alveolar Damage or Destruction

The regeneration and healing of the lung alveoli by L-NIL is shown in a mouse model of intoxication by chronic inhalation of tobacco smoke. In this model, mice are exposed to a stream of tobacco smoke in a concentration of 140 mg/m³ for 6 h per day, 5 days a week over a period of up to 8 month. It is demonstrated that mice, which are kept under these conditions show within 8 month structural and functional destruction of alveolar structure and therefore loss of lung function.

The structural and functional pulmonary changes due to toxic smoke exposure were quantified with techniques suitable for this purpose. To assess the degree of alveolar damage, the mean linear intercept, alveolar septal wall thickness and the air space volume was quantified.

Parameters for the extent of vascular remodeling comprise the measurement of vascular wall thickness, staining for alpha-smooth muscle actin and von-Willebrand factor as well as the mean cross-sectional area of the vessels.

Functional measurements include the quantification of the lung function in spontaneously breathing as well as artificially ventilated mice with respect to airway resistance, lung compliance and volume flow. To evaluate functional changes of the vessels, lungs are investigated in an isolated perfused and ventilated experimental set-up, and pulmonary vascular resistance and reactivity are measured. The vascular reactivity is determined by quantifying the extent of vasoconstriction after addition of phenylephrine and can be deduced from dose/effect curves.

The positive effect of L-NIL on the lungs of mice is in parallel also demonstrated by oral administration of L-NIL in the drinking water (e.g. in a concentration of 600 μg/ml). In these experiments, a complete protection against alveolar damage/destruction became evident as demonstrated via alveolar morphometry and lung functional measurements. L-NIL treatment resulted in a complete protection against the occurrence of alveolar destruction (as demonstrated by hemodynamic measurements, heart and vascular morphometry).

The restoration of the structural and functional impairments of the damaged/destroyed alveoli induced by exposure of toxic smoke for eight months is of direct clinical importance.

In a second experiment, L-NIL was administered orally via drinking water in a concentration of 600 μg/ml for a period for 3 months to treat mice with alveolar damage/destruction as a result of 8 months of toxic smoke exposure. A complete regeneration of the alveolar lung structure was achieved as well as lung function improvement. In sharp contrast, placebo-treated mice showed no signs of improvement.

What is claimed is:

1. A pharmaceutical composition for the treatment of pulmonary alveolar damage and destruction comprising N⁶-(1-iminoethyl)-L-lysine.

2. The composition of claim 1, wherein the composition is used for the treatment of mammals.

3. The composition of claim 2, wherein the composition is used for the treatment of humans.

4. The composition of claim 1, wherein the composition is administered orally, by inhalation or by injection.

5. The composition of claim 1, wherein the N⁶-(1-iminoethyl)-L-lysine is present as a salt, ester or amide.

6. A method of treatment of pulmonary alveolar damage or destruction, the method comprising, administering to a patient in need thereof, a therapeutically effective amount of a pharmaceutical composition comprising N⁶-(1-iminoethyl)-L-lysine or a salt or ester, or amide thereof, wherein administration of the pharmaceutical composition treats said pulmonary alveolar damage or destruction.

7. The method of claim 6, wherein the pharmaceutical composition is administered to mammals such as humans.

8. The method of claim 6, wherein the pharmaceutical composition is administered orally, by inhalation or by injection.
